(19) United States
(12) Patent Application Publication
(10) Pub. No.: US 2007/0016219 A1
(43) Pub. Date: Jan. 18, 2007

(54) Vertebral Marker Devices and Installation Methods

(76) Inventor: Marc J. Levine, Richboro, PA (US)

Correspondence Address: CASTELLANO MALM PLLC, 1250 CONNECTICUT AVENUE NW, SUITE 200, WASHINGTON, DC, 20036, US

(21) Appl. No.: 11/457,527

(22) Filed: Jul. 14, 2006

Related U.S. Application Data
(60) Provisional application No. 60/698,921, filed on Jul. 14, 2005.

(51) Int. Cl. A61F 2/00

(52) U.S. Cl. 606/99

(57) ABSTRACT

A cannulated marker device for use in a spinal fixation procedure, particularly for insertion of pedicle screws, is disclosed. The marker device is configured to be inserted over a guide wire or directly with an insertion device into the spinal bone at a desired location, and left in place without interfering with subsequent steps of the spinal fixation procedure. Also provided, is an insertion device comprising a shaft attached to a handle at a first end, and a second end being configured to be detachably attached to a marker device of the present invention. A method for marking a location on a patient's spinal column is also provided in connection with the marker device and the insertion device of the present invention.

### CROSS-REFERENCE TO RELATED APPLICATIONS

The present application claims priority to U.S. Provisional Patent Application Ser. No. 60/698,921, filed Jul. 14, 2005, the entire contents of which are incorporated herein by reference.

### FIELD OF THE INVENTION

The present invention generally relates to a system and method for marking a location on a vertebral body, such as a pedicle, for assisting in placement of pedicle screws in the spine.

### BACKGROUND OF THE INVENTION

The spine is vulnerable to injury and to degeneration (gradual failure with aging and wear/tear), and diseases of the spinal column can occur on many levels. The structures most vulnerable to degeneration are the soft tissues, namely the intervertebral discs, the ligaments and cartilage of the facet joints. The discs gradually collapse, the facet joints thicken and ligaments lose their elasticity and stabilizing ability. Degeneration has a tendency to lead to settling between vertebrae (sometimes also shifting, such as in spondylolisthesis), narrowing of the spinal canal and neuroforamen at the affected levels (usually lower lumbar spine), and a loss of space for the nerve elements (spinal stenosis). Thus, one of the most common surgical procedures performed on the spine thus involves spinal decompression, i.e. freeing up the compressed nerves.

As decompression removes tissue of the spinal column, it weakens the spinal column. Therefore, fusion is often necessary for spinal stability following a decompression procedure. In simplest terms a spinal fusion is a growing together of bone structures creating a solid bone bridge between vertebrae. Many fusion techniques are available, depending upon a number of factors including: level(s) to be fused, degree of instability or deformity, age of the patient, risk factors for non-union (failure to fuse properly), and experience of the surgeon. Some of the common basic types of fusions include anterior spinal fusion, posterior fusion without spinal instrumentation, posterior spinal fusion with instrumentation, circumferential fusion (anterior and posterior), circumferential fusion from posterior only, which has two common versions, PLIF (posterior lumbar interbody fusion) and TLIF (transforaminal lumbar interbody fusion).

However, following the surgical procedure, fusion takes additional time to achieve maximum stability and a spinal fixation device is typically used to support the spinal column until a desired level of fusion is achieved. Depending on a patient's particular circumstances and condition, a spinal fixation surgery can sometimes be performed immediately following decompression, without performing the fusion procedure. The fixation surgery is performed in most cases because it provides immediate postoperative stability and, if fusion surgery has also been performed, it provides support of the spine until sufficient fusion and stability has been achieved.

Conventional methods of spinal fixation utilize a rigid spinal fixation device to support an injured spinal part and prevent movement of the injured part. These conventional spinal fixation devices include: fixing screws configured to be inserted into the spinal pedicle or sacral of the backbone to a predetermined depth and angle, rods or plates configured to be positioned adjacent to the injured spinal part, and coupling elements for connecting and coupling the rods or plates to the fixing screws such that the injured spinal part is supported and held in a relatively fixed position by the rods or plates.

Locating the entry point for the pedicle screw is one of the most challenging aspects of performing spinal fixation procedures. Traditionally, an “open” technique is used for the placement of the fixing screws (or pedicle screws), which requires a rather extensive midline incision and dissection to find the starting entry point for a screw. This technique is traumatic and increases patient recovery time. Surgeon experience usually dictates as to whether or not X-ray assistance is needed in the operating room for placement of screws. The screws used in the open technique are not cannulated. Most open fusion procedures also incorporate a decompression of nerves or laminectomy. In this procedure the decompression is usually done prior to screw placement. Anatomical landmarks are used to find the entry point, but confined working space and bleeding due to removal of soft tissues make accurate location difficult. The accuracy of screw placement with this open technique can be as low as 70%. Finally, a fusion is performed after the screws are placed and outside (lateral to the screws).

More recently spine surgery has seen a shift from open procedures to minimal invasive techniques. Recently, a procedure called vertebroplasty or kyphoplasty has gained wide spread acceptance, and has made spine surgeons very comfortable with accessing pedicles percutaneously using intraoperative fluoroscopic imaging. Kyphoplasty is a minimally invasive procedure to alleviate pain from vertebral compression fractures, where an orthopedic balloon is placed in the affected vertebra and inflated. The resulting cavity is filled with bone cement in order to stabilize the vertebral fracture.

Screws can be placed percutaneously through mini-incisions and using two fluoroscopy machines showing anterior/posterior (AP) and lateral images on request to monitor the screw placement. These screws are typically cannulated and are placed over a guide wire introduced into the pedicle with the assistance of contemporaneous intraoperative images. Once the guide wire is accurately placed, the screw goes over the guide wire and the wire is removed. However, because few cases are amenable to percutaneous decompressions, currently most percutaneous screws are placed in patients who do not need decompressions. Furthermore, it would not be practical to place the screws percutaneously and then do the decompression, because the screws would hinder the open decompression of nerves.

U.S. Pat. App. No. 2005/0065515 discloses a marking and guidance device for marking a desired location on the spinal pedicle where a pedicle screw will be inserted and guiding the pedicle screw to the marked location using a minimally invasive surgical technique. The device uses a tubular hollow guider which receives within it an inner trocar having a sharp tip at one end that penetrates a patient's muscle and tissue to reach the spinal pedicle. The trocar is in the form of a long tube or cylinder having a diameter smaller than the inner diameter of the hollow of the guider so as to be inserted into the hollow of the tubular guider. The trocar further includes a sharp or pointed tip for penetrating the vertebral body through the pedicle. The system further employs fiducial pins to be inserted into the hollow of the guidance tube and thereafter be fixed into the pedicle.

The fiducial pins are not hollow or cannulated and have a cylindrical shape with a diameter smaller than the inner diameter of the hollow of the guider tube in order to pass through the hollow of the guider. The fiducial pin is pushed into the pedicle by a pushing trocar. After the fiducial pin is inserted into the spinal pedicle, a larger hole or area centered around the pin is created to allow easer insertion and mounting of a pedicle screw into the pedicle bone. The larger hole is created using a cannulated awl inserted over the fiducial pin fixed at the desired position of the spinal pedicle. After the cannulated awl has created a larger insertion hole for the pedicle screw, the fiducial pin is optionally removed.

The fiducial pin disclosed in this patent application, however, is a fine, non-hollow device. Once placed in position, it must always be under the direct view of the surgeon or it would be very difficult to locate them if the surgeon should ever lose sight of it. In addition, subsequent operative steps, especially the insertion of the pedicle screw with the pin left in place, post a serious risk that the fiducial pin may be caused to be inserted further into the spine bone, penetrating the spinal cord or pushed through the vertebral body into structures that sit in front of the spine in the abdominal and chest cavity and causing serious injuries to the patient.

Accordingly, there is a need for an improved method and system for marking a location of a vertebral body and assist pedicle screw placement.

### SUMMARY OF THE INVENTION

One embodiment of the invention is generally directed to a marker device for use in a spinal fixation procedure. The marker device comprises a cannulated body configured and dimensioned to pass over a guide wire and be inserted into the spinal bone and left in place to visually mark a location where a screw can thereafter be inserted into the spine bone. In one embodiment, the body includes a shaft portion and a head portion. The shaft portion is configured and dimensioned to be inserted into bone and the head portion is configured and dimensioned to remain external to bone. The shaft portion has a first outer diameter and the head portion has a second outer diameter and the second outer diameter is larger than the first outer diameter. Also provided, is an insertion device comprising a shaft attached to a handle at a first end, and a second end being configured to be detachably attached to a marker device of the present invention. A method for marking a location on a patient's spinal column is also provided in connection with the marker device and the insertion device of the present invention.

### BRIEF DESCRIPTION OF THE DRAWINGS

FIGS. 1A-1C depict a side view, a side cross-sectional view and a top view, respectively, of one embodiment of a marker device of the present invention;

FIGS. 2A-2B show a perspective view of one embodiment of an insertion device of the present invention and an enlarged view with a marker device attached to the insertion device in use about a guide wire;

FIGS. 3A-3D depict another embodiment of an insertion device having a trocar device attached to the tip;

FIGS. 4A-4D illustrates the insertion device depicted in FIGS. 3A-3D in combination with one embodiment of a marker device; and

FIG. 5 depicts alternate embodiment of marker devices of the invention having alternative tip configurations.

### DETAILED DESCRIPTION OF THE INVENTION

The present invention takes advantage of the current comfort level of placing guide wires into pedicles percutaneously with intraoperative radiological (e.g. X-ray) guidance. The present invention is generally directed to marker devices, and related techniques and tools for its application. Various embodiments of the marker devices may be provisionally percutaneously placed in a pedicle to mark the pedicle and may be referred to as “3PM” devices. According to some embodiments, the devices and methods of the present invention assist surgeons in the placement of pedicle screws in patients, and may be particularly well suited for use in patients that may require nerve decompression. In other embodiments, the marker devices may also be used as an access portal to a vertebral body for insertion of other materials, including, but not limited to drugs such as antibiotics or other medicaments, bone cement, or bone morphogenic proteins.

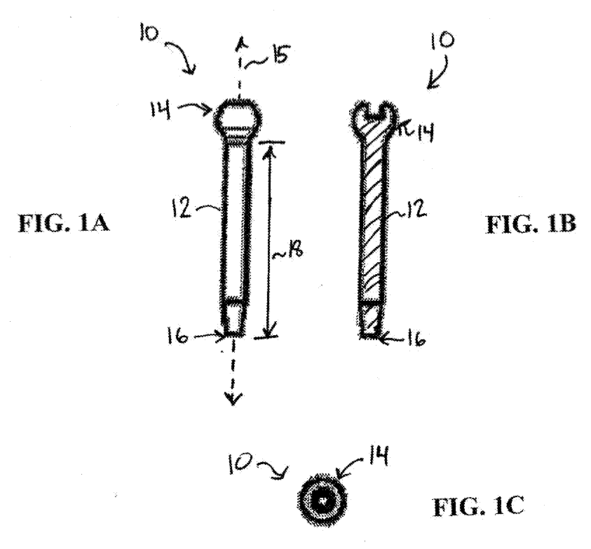

Referring to FIGS. 1A-1C, one embodiment of a marker device 10 according to the invention generally comprises a cannulated shaft 12 with a generally spherical or ball shaped head or proximal end 14. Shaft 12 extends longitudinally along axis 15 from a tip or distal end 16 a length 18 to proximal end 14. The length 18 of shaft 12 may vary to accommodate the various lengths of different pedicles of a patient. The ball shaped proximal end 14 generally prevents the marker device 10 from being inadvertently pushed too far into the pedicle. Proximal end 14 also facilitates connection of the marker device 10 to an insertion device 20 (described below) as the inner portion of proximal end 14 may be threaded or otherwise configured to receive the insertion device 20. According to one embodiment, the ball shaped head 14 may have a smooth external surface. In another embodiment, shaft 12 may also have a smooth outer surface to facilitate being pushed into bone. Also, such smooth exterior surfaces may facilitate easy and non-traumatic retraction of tissue and further allow for access of additional lateral structures. According to one embodiment, marker device 10 is generally configured to have a low profile so as to not interfere with any dissection that may be carried out prior to the insertion of pedicle screws. As one skilled in the art may appreciate, placement of pedicle screws too early in a surgical procedure can be an obstruction. According to another embodiment, the ball shaped head 14 may serve as a pivot point for a circular reamer device that can spin on the ball to remove adjacent bone, making more room for the larger head of a pedicle screw that will eventually sit where the ball head is. In another variation, shaft 12 may be threaded to allow for tapping the path of the eventual larger diameter screw placement. In this regard, such a feature may save a step for the surgeon later on. According to one embodiment, a tip portion of shaft 12 adjacent distal end 16 may be tapered, sharpened or otherwise configured to facilitate entry into bone.

According to one embodiment, marker device 10 may be made of any suitable radio-opaque material such that the marker device is visible in an X-ray device. According to one embodiment, marker device 10 is made from a rigid biocompatible metal or synthetic material.

As one skilled in the art can appreciate, marker device 10 can be utilized with multiple different types of known cannulated screw systems. In this regard, marker device 10 generally facilitates precise placement of pedicle screws for any situation where X-ray can be used to locate a pedicle followed at some point by placement of the pedicle screw under direct visualization.

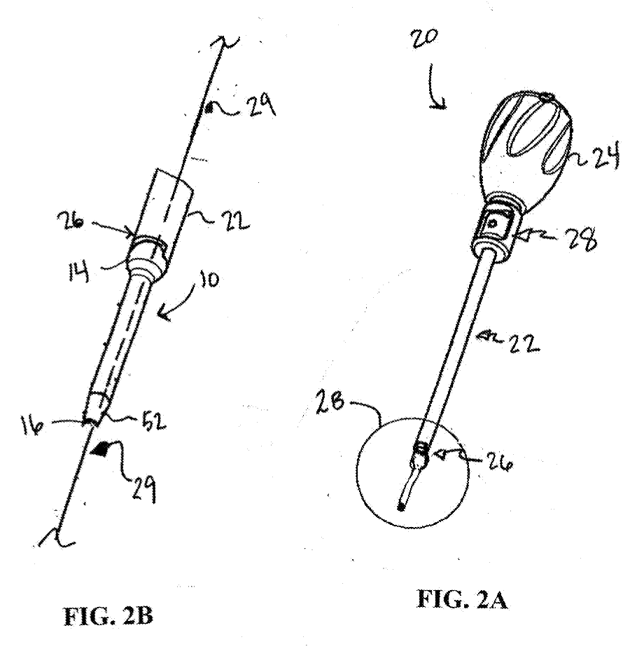

Referring to FIGS. 2A-2B, one embodiment of an insertion device 20 is shown. Insertion device 20 generally comprises an elongate cannulated shaft 22 extending distally from a handle 24. Distal tip 26 of shaft 22 is generally configured and dimensioned to engage proximal end 14 of marker device 10 such that rotational and axial forces may be imparted upon marker device 10 when connected to insertion device 20. In this regard, distal tip 26 may have any suitable mechanical linkage sufficient to impart such forces, including but not limited to a threaded connection mechanism. A rotational wheel 28 may be provided intermediate shaft 22 and handle 24 to control the threaded engagement or connection of distal tip 26 to proximal end 14 of marker device 10. As best seen in FIG. 2B, according to this embodiment, insertion device 20 may travel along and marker device 10 may be installed over a guide wire 29. Guide wire 29 may be placed into a pedicle using known techniques. According to one method, two fluoroscopic imaging machines may be used with a minimally invasive technique to guide the guide wire into place. A cannulated marker device 10 of the present invention is then placed over guide wire 29 and advanced to the pedicle site and inserted into the pedicle with the ball shaped head 14 flush to the bone or with the proximal end 14 slightly elevated above the bone or spaced from the bone. According to one variation, the length 18 of the marker device may be shorter than the pedicle screw which will take its place. As discussed above, the marker device 10 may have a smooth outer surface which is driven down with an insertion device or configured as a “tap” and screwed into place with the insertion device 20.

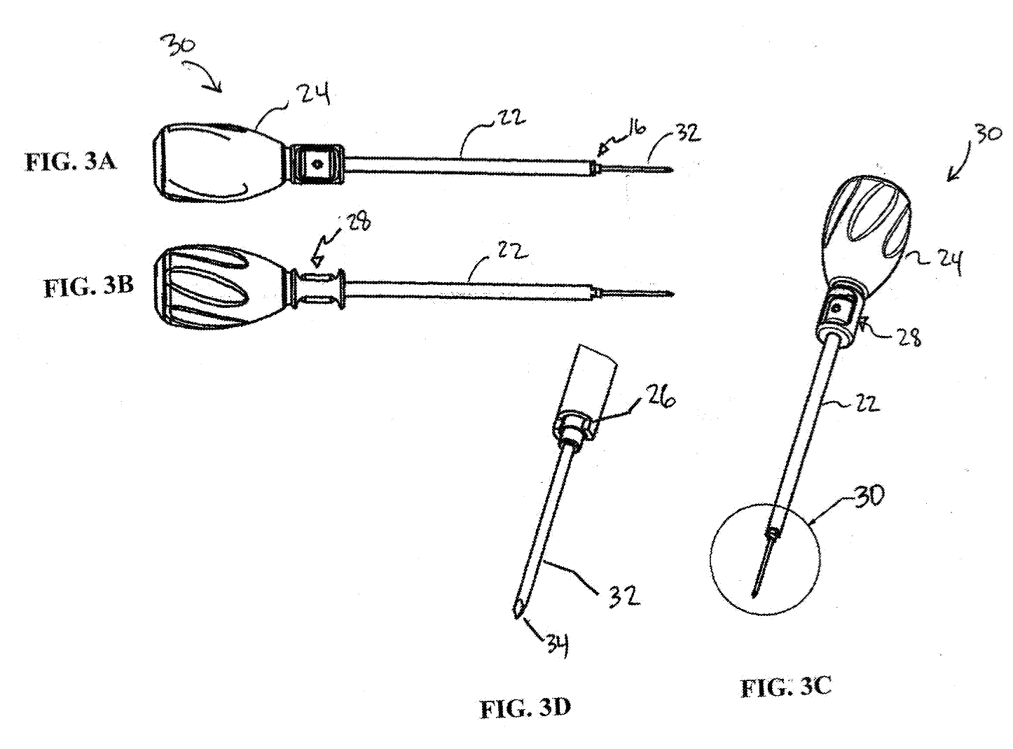

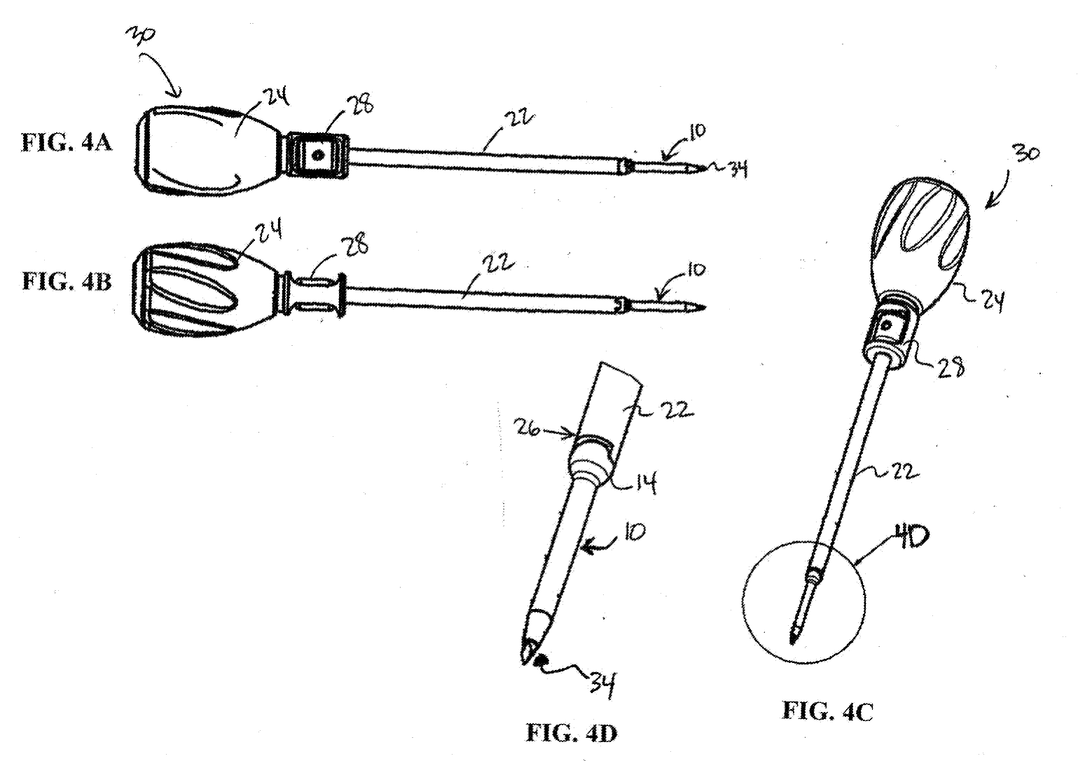

Referring to FIGS. 3-4, according to another embodiment of the invention, a “one-step” process is also provided, wherein the marker device 10 is inserted with an alternative insertion device 30 without the use of a guide wire. As best seen in FIGS. 3A-3D, according to this embodiment, insertion device 30 generally comprises an elongate trocar 32 extending from a distal end thereof that is coaxially received through the cannula of marker device 10 when it is attached to the end of the insertion device. As best seen in FIG. 4D, a tip portion 34 of trocar 32 extends through the distal tip of marker device 10. Tip portion 34 of trocar 32 is generally sharp and is configured to penetrate bone to facilitate insertion of marker device 10 directly into the desired location on the pedicle. In this regard, utilization of insertion device 30 eliminates the need for use of a guide wire during placement of marker devices 10.

According to the above described embodiments, after the placement of the marker device 10, the insertion device 20, 30 is removed and the marker device 10 is left in place. If a guide wire 29 is used, it is also removed.

The above steps are repeated until each of the desired number of screw sites is marked by a marker device.

Once the marker devices are installed, a surgeon may perform one or more suitable operative procedures at or near the marked locations. For example an open decompression of nerves may be performed. Once the open decompression is completed the surgeon may, for example, perform a bony fusion outside of where the markers are placed, or other surgical procedures. In general, a low profile marker device of allows easy access to these areas. In contrast, certain prior art methods place full screws into the pedicle prior to performing the bony fusion, rendering access to these areas difficult.

Following suitable operative proceedings, a guide wire may then be placed through the marker device 10 and the marker device may then be removed over the guide wire.

According to one embodiment, a cannulated pedicle screw may then be placed over the guide wire. Once the pedicle screw is in place, the wire is removed and the screw left in place.

The above steps of screw placement and wire removal may be repeated for each marker device that is placed.

Once all screws have been placed the surgeon may then attach rods or plates or other suitable parts of a spinal fixation device to complete the fusion.

The devices and methods of the present invention generally allow for a more accurate placement of screws using X-ray guidance or other suitable radiological techniques. According to one aspect, the devices and methods takes advantage of the ease of percutaneous technique for wire placement and high confidence with using hi-planar fluoroscopy. Marker devices 10 may also be placed prior to the surgical procedure under CAT scan guidance by a radiologist and then easily found by the surgeon intraoperatively. Such a technique may be particularly advantageous if severe deformities exist or for more challenging pedicles such as in the thoracic spine.

The device and method of the present invention generally decrease the risk of infection since the amount of time needed for a large open wound would be minimized as the pedicles are prepared before the incision is ever made. In addition, compared to the traditional screw placement methods with open incisions, which are often assisted by fluoroscopic machines placed over the open incision to provide an anterior-posterior view, the devices and methods of the present invention generally does not require such an anterior-posterior view. This also decreases the risk of infection, and decreases the amount of radiation exposure of the patient.

Traditional screw placement with open incisions requires a fairly long incision to get adequate exposure and extensive tissue stripping to find the correct starting point based on intra-operative anatomy. The present invention marks the pedicle/screw with the marker device prior to making any large midline incision and performing the decompression, and allows for a smaller incisions and less tissue stripping, particularly of facet joints, thus minimizing trauma to the patient and improves patient recovery time.

Furthermore, the size of the incision for the percutaneous portion of this procedure (that is, the placement of the marker device) is smaller than that needed for percutaneous pedicle screw placement. Both the placement of the insertion device and placement of the guide wire generally cause minimal bleeding. Both of these are additional reasons not to place the screws percutaneously for open procedures.

The device and related method of the present invention is suitable to be used with screws with various configurations that are currently available commercially for percutaneous placement of screws. It is universally applicable for the placement of cannulated screws. In alternative embodiments, screws need not be inserted. In this regard, the marker devices may also be used as an access portal to a vertebral body for insertion of other materials, including, but not limited to drugs such as antibiotics, bone cement, or bone morphogenic proteins.

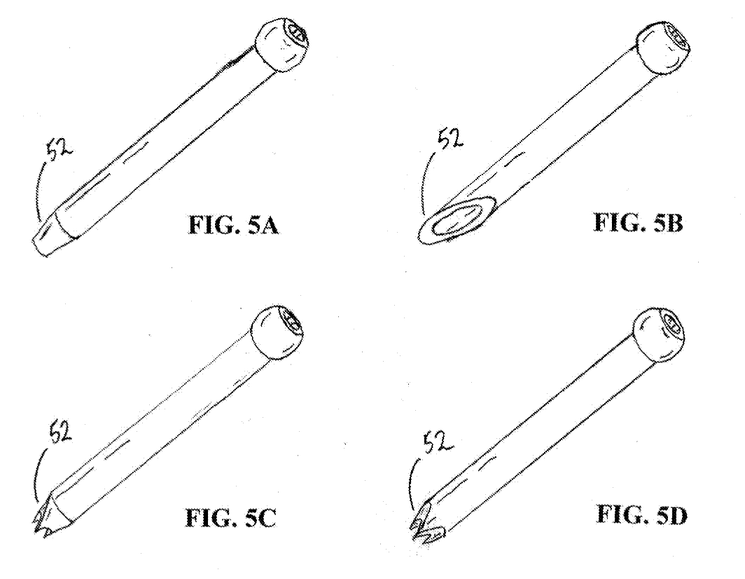

Referring to FIGS. 5A-5D, in alternative embodiments, the tip portion 52 of shaft 12 of marker devices 10 can have varied shapes and configurations. For example, tip portion 52 may have a generally conical taper as shown in FIG. 5A. As shown in FIG. 5B, tip 52 may alternatively have a bevel. In certain other embodiments, shown in FIGS. 5C and 5D, a plurality of points may be spaced about the perimeter of distal end 16.

### EXAMPLES

### Example 1

### Operating Procedure Using a Marker Device

A patient is positioned properly for the operation, e.g. on a table. Proposed pedicles for receiving pedicle screws are located under fluoroscopic guidance, generally requiring anterior/posterior and lateral views.

A. Placement of the Marker Device Using a Guide Wire

A needle is used to poke through the skin and down to the bone where the desired entry point is on the pedicle with the X-ray guidance. A small incision may be made for insertion of a jam sheidy needle.

Following the incision, the jam sheidy or other trocar/sleeve device is inserted into the patient through the incision. The insertion of the trocar device through the bone and pedicle and into a vertebral body is monitored using X-ray visualization. Once the trocar is appropriately placed, the trocar tip is removed, thus leaving the trocar sleeve or tube in place.

A guide wire is placed through the trocar sleeve and pushed into the vertebral body, while being monitored with a lateral X-ray view.

Once the guide wire is in place the trocar tube is removed. At this point a wire should be in the vertebral and sticking through the skin.

A marker device is attached to an insertion device. Both the marker device and insertion device are cannulated so that they can slide over the guide wire that is sticking through the skin.

Again, the marker device may take the form of a smooth device requiring gentle and controlled impaction, or it may be in the form of a tap and therefore can be screwed into the bone over the wire. A screwed-in marker device has the advantage of allowing easier insertion of the larger pedicle screw later on.

The marker device attached to the insertion device is then passed over the guide wire. Lateral X-ray view is used to ensure that the marker device proceeds appropriately and that the wire which it is going over is not being driven out the front of the vertebral body which could cause injury to the patient.

With the insertion device, the marker device is driven down until the head portion (e.g. a ball head portion) hits the bone which can be felt by the surgeon and/or seen on lateral X-ray. At this point, the guide wire is removed, simply by pulling it out through the cannulated insertion device and the marker device. The insertion device is then detached from the marker device and removed, and the marker device is left in place until it is time to exchange it for a pedicle screw.

At this time a surgeon can perform the remainder of the spine operation including perhaps the decompression and preparation for a fusion. After the desired operating procedures are performed, a surgeon may then replace one, several, or all of the marker devices with a pedicle screw, as desired. According to one embodiment, the screws are not placed percutaneously, but rather thru a larger incision such as the one used for performing a decompression, for example. In contrast, the incision required for a percutaneous screw placement is generally much larger than that required for placement for the marker device. For percutaneous screws to be placed, typically a series of skin and soft tissue dilators are required to allow a screw to pass through the tissue.

Once it is time to place the actual pedicle screw, a guide wire is placed back through the installed marker device under direct visualization. One skilled in the art will appreciate that a pedicle marker device according to the present invention generally requires nothing more than a stab incision thru the skin to get placed. The guide wire may be scored with measurement marks which allow one to determine how deep it is with relation to the fixed length of the marker device. This allows the surgeon to avoid pushing the wire too far. Some surgeons may prefer using lateral X-rays only for additional assistance, but there is no need for anteroposterior X-rays.

The insertion device is placed over the guide wire and reattached to the marker device to remove the marker device. That is, the insertion device may also function as a removal device. As the guide wire is longer than the insertion device with the marker device attached, the wire remains visible outside the skin. The marker device is removed leaving a wire in place.

A cannulated pedicle screw may then be placed over the wire. Many such pedicle screws are available commercially, and the configuration and the length of the screw can be based on the location of the marker device seen on a lateral X-ray.

Once the screw is installed in the pedicle, the wire is removed and the screw is left in place.

The above technique for percutaneous placement of a cannulated marker may also be used for the placement of a cannulated screw with a traditional midline open procedure and the foregoing procedure is not limited to a percutaneous approach.

For example, as an alternative, the marker devices can be used in a completely open procedure under direct visualization with or without X-ray guidance. For example, certain intraoperative circumstances may arise during a spinal surgical procedure where a surgeon finds a spine to become unstable and require a fusion that otherwise was not planned for or the need for such could not be determined until after a decompression for instance in a trauma setting. The marker device and instrumentation is not nearly as cumbersome as traditional devices used for pedicle screw insertion particularly when used under the limited space created by fluoroscopic imaging devices. For this reason surgeons may find it both easier and safer to place pedicle screws with the assistance of the above-described marker devices and techniques. The devices and instrumentation are configured and dimensioned to be non-cumbersome that can easily be used under an X-ray machine which may be over the wound. One skilled in the art will appreciate that the aforementioned provisional location of the pedicle facilitates precision and allows delicate maneuvering as compared to other techniques.

Finally, one skilled in the art can appreciate that this technique in conjunction with the marker devices of the invention may be very helpful for surgeons performing a Wiltse approach for placement of pedicle of screws. Certain spine surgical procedures require a Wiltse approach. In this approach the traditional midline incision to the spine is substituted for by lateral incisions directly over the lateral elements of the vertebrae such as the facet joint and transverse processes. For instance one may want to extend a fusion. To accomplish this, the previous rod would need to be removed and additional screws placed with subsequent placement of a longer rod which would then include the previous screws and also the additional screws. In this situation a midline incision may force a surgeon to go through dangerous scar tissue. The well-established Wiltse (lateral incision) approach however would directly come down on the previous instrumentation. Use of the marker device would allow accurate placement of additional screws with all its advantages as described previously. In addition placement of the marker devices would help determine the length and location of the incision. By percutaneously determining the pedicle the location of other structures needed to extend the fusion such as the transverse processes can easily be found with minimum dissection and tissue stripping thereby decreasing operative time, infection and complications.

B. Placement of the Marker Device Without Using a Guide Wire

As an alternative, the marker devices of the invention may also be inserted without the use of a guide wire. After an initial incision, an insertion device with a marker device attached is inserted directly into the patient, placing the marker device suitably at the location on the bone. The insertion device is then detached and removed. All other steps are essentially the same as the ones described above for the method using a guide wire, and are performed with the assistance of X-ray visualization. This “one-step” technique, whereby a guide wire is not utilized, may also be used in a completely open procedure under direct visualization with or without X-ray guidance.

What is claimed is:

1. A marker device for use in a spinal fixation procedure to mark a location on or near a patient's spinal bone, comprising:
a cannulated body configured and dimensioned to pass over a guide wire and be inserted into the spinal bone and left in place to visually mark a location where a screw can thereafter be inserted into the spine bone,
the body comprising a shaft portion and a head portion, wherein the shaft portion is configured and dimensioned to be inserted into bone and the head portion is configured and dimensioned to remain external to bone, and wherein the shaft portion has a first outer diameter and the head portion has a second outer diameter and the second outer diameter is larger than the first outer diameter.

2. The marker device according to claim 1, wherein the body is made of a rigid biocompatible metal or synthetic material.

3. The marker device according to claim 1, wherein the shaft portion extends a first length along a longitudinal axis from a distal tip to the head portion, and wherein the first outer diameter is smaller and the first length is shorter than that of a pedicle screw to be inserted into the location occupied by the marker device.

4. The marker device according to claim 1, wherein the shaft portion has a smooth outer surface to facilitate being pushed into bone.

5. The marker device according to claim 1, wherein the shaft portion has a threaded outer to facilitate being screwed into bone.

6. The marker device according to claim 1, wherein the shaft portion has a tip portion adjacent a distal end, wherein the tip portion is tapered, sharpened or otherwise configured to facilitate entry into bone.

7. The marker device according to claim 1, wherein the head portion is configured to receive an insertion device for inserting the marker device into bone and retrieving the marker device from bone.

8. The marker device according to claim 7, wherein the head portion has a smooth outer surface for non-traumatic retraction of tissue thereover.

9. An insertion device comprising a first shaft attached to a handle at a first end, and a second end configured to be detachably attached to a marker device of claim 1.

10. The insertion device according to claim 9, wherein the second end is figured to have a sharp tip that extends through and protrudes out of a distal end of the marker device for creating a path for one step insertion of the marker body.

11. A method for marking a location on a patient's spinal column where a spinal fixation procedure is to be performed, and for guiding the insertion of a cannulated pedicle screw of a spinal fixation device, the method comprising:
(1) locating an entry point where the pedicle screw is to be inserted with the assistance of a radiological instrument, and
(2) inserting a marker device according to claim 1 into a spinal bone at the entry point percutaneously, wherein the marker device is left in the spinal bone at the entry point, marking the entry point and the location.

12. The method according to claim 11, wherein prior to inserting the marker device into the spinal bone, a guide wire is inserted into the spinal bone at the entry point and the marker device is inserted over the guide wire.

13. The method according to claim 11, wherein the shaft portion of the marker device is inserted completely such that head portion is flush to the bone.

14. The method according to claim 11, wherein the marker device is inserted into the spinal bone with the head portion spaced from the bone.

15. A method for performing a spinal fixation procedure which includes at least one of spinal decompression, spinal fusion, and stabilization, the method comprising marking an entry point according to claim 11 or claim 12 and performing the spinal procedure at or near the location marked by the marker device.

16. A method for inserting a cannulated pedicle screw, comprising marking an entry point according to claim 11, and further:
(3) inserting a guide wire into the spinal bone through the marker device, such that the guide wire marks the entry point and is visible outside the skin;
(4) retrieving the marker device over the guide wire, and
(5) inserting the cannulated pedicle screw over the guide wire.

17. The method of claim 16, wherein an enlarged entry hole centered around the marker device or the guide wire is created to facilitate insertion of the pedicle screw into the vertebral bone.

18. A method for performing a spinal fixation procedure which includes at least one of spinal decompression, spinal fusion, and stabilization, the method comprising inserting at least two cannulated pedicle screws according to claim 16 and further securing a spinal fixation device to the pedicle screws.

19. The method according to claim 18, where the spinal fixation device is secured using an open procedure without fluoroscopic guidance.

20. The method according to claim 11, wherein the cannulated marker defines an access portal into the vertebral body, wherein a medicament may be inserted through said portal into the vertebral body.

21. The method according to claim 11, wherein bi-planar fluoroscopy is used to facilitate the placement of the marker device.

22. The method according to claim 18, wherein CAT Scan is used to facilitate at least one step selected from the group consisting of initial placement of the guide wire, placement of the marker device, re-insertion of the guide wire, and retrieval of the marker device after insertion of the pedicle screw.

23. A marker device for use in a spinal fixation procedure to mark a location on or near a patient's vertebrae, comprising:
a cannulated body configured and dimensioned to be inserted into the spinal bone and left in place to visually mark a location of a vertebral body,
the cannulated body comprising a shaft portion and a head portion, wherein the shaft portion is configured and dimensioned to be inserted into bone and the head portion is configured and dimensioned to remain external to bone, and wherein the cannulated body is configured and dimensioned to receive a trocar extending coaxially therethrough,
wherein a tip portion of the trocar is generally sharp and is configured to penetrate bone to facilitate insertion of the marker device directly into a desired location on the vertebral body.
